(19) United States
(12) Patent Application Publication
(10) Pub. No.: US 2025/0017450 A1
(43) Pub. Date: Jan. 16, 2025

(54) INSPECTION ROBOT

(71) Applicant: UNIVERSITY OF DUNDEE, Dundee (GB)

(72) Inventors: Alfred CUSCHIERI, St Andrews (GB); Hamza KHAN, Dundee (GB)

(21) Appl. No.: 18/290,771

(22) PCT Filed: Jul. 18, 2022
(86) PCT No.: PCT/EP2022/070055

§ 371 Date: Jan. 20, 2024

(30) Foreign Application Priority Data
Jul. 22, 2021 (GB) 2110576.2

(51) Int. Cl. A61B 1/00; A61B 5/00

(52) U.S. Cl. CPC A61B 1/00156; A61B 1/00032; A61B 1/0016; A61B 5/4255; A61B 5/6873

(57) ABSTRACT

An inspection robot for inspecting gastrointestinal tracts or other lumen. The inspection robot is capable of locomotion and comprises: a body, a vibration actuator, and a plurality of resilient legs. The plurality of resilient legs are arranged to protrude outwardly and rearwardly from the body, with respect to a direction of locomotion. Each leg is coupled to the vibration actuator at a proximal end of the leg and the vibration actuator is operable to induce vibrations in a distal end of the leg, which serve, in use, to propel the body in the direction of locomotion by the distal end generating a pushing force against an external surface.

### TECHNICAL FIELD OF THE DISCLOSURE

The present disclosure relates to an inspection robot capable of locomotion, and in particular to an inspection robot for gastrointestinal tracts or any other lumen.

### BACKGROUND

Direct visual examination of an inner surface of a lumen—e.g. gastrointestinal tracts (GIT)s or other lumen—is a critical tool for diagnosing a wide range of disorders.

Conventionally, direct visual inspection is performed using an endoscope. The endoscope comprises a long, thin, flexible tube with a camera and light source at one end. The endoscope is inserted through a natural bodily orifice and thereafter is steered to an inspection site of the GIT or any other lumen (i.e. for a complete inspection of the GIT or other lumen).

Disadvantages of conventional endoscopy include patient discomfort and a need for highly trained operators (i.e. endoscopists). Additionally, steering and manipulating the endoscope, especially within narrow or tortuous bodily cavities, is mechanically difficult and may result in tenting of the lumen and/or looping of the endoscope. Both tenting and endoscope looping significantly increases patient discomfort and a risk of damage to the lumen (e.g. including bowel wall perforation).

A small intestine (SI) is a narrow, coiled, and tortuous tube that occupies a central part of a patient's abdominal cavity. Accessing the SI using an endoscope is usually performed through the patient's mouth. Accessing the SI through the mouth is difficult because of a complex route that the endoscope must be steered through to get to the inspection site (i.e. through a pyloric sphincter, a duodenum, a jejunum and the rest of the SI down to a caecum).

Capsule endoscopy inspection robots (i.e. capsule endoscopy inspection pills) have been developed to enable direct visual examination of the GIT without the need for highly trained operators. The inspection robot is swallowed by the patient and passes passively (i.e. assisted by peristaltic contractions of the GIT) through the patient's oesophagus and stomach of the GIT. The inspection robot inspects and wirelessly transmits GIT images to the endoscopist (i.e. via a data logger worn by the patient) or stores the images using on-board memory to be viewed by the endoscopist after the inspection robot exits the patient via the anus (i.e. the images are loaded onto a computer from the on-board memory for viewing by the endoscopist). Such inspection robots create less patient discomfort compared to flexible conventional endoscopes and present no risk of tenting of the lumen or endoscope looping (although inspection robots may cause other complications including retention).

Disadvantages of inspection robots include limited battery life and an inability for some patients to swallow the inspection robot (e.g. because of inspection robot size and rigid body). Additionally, current inspection robots lack controlled locomotion (i.e. movement). Therefore, current inspection robots rely on gravity or passive propulsion (peristalsis assisted by contractions of muscular GIT walls) to reach the inspection site. The lack of controlled locomotion is a serious disadvantage of current inspection robots. For example, because the endoscopist is unable to orient the capsule for close inspection of the inspection site and/or the endoscopist is unable to back-up the inspection robot (e.g. for opportunistic inspection of previously undetected suspect lesions).

Attempts have been made to develop inspection robots with controlled active locomotion. For example, magnetically guided inspection robots rely on a complex magnetic guidance system.

Other attempts to develop inspection robots with controlled locomotion rely on actively powered locomotion using mechanical moving parts (e.g. paddles), which generate propulsion by exerting friction on intestinal mucosa. Such actively powered locomotion carries an innate risk of damaging the GIT (i.e. the intestinal mucosa) and inducing bleeding and GIT perforation.

It is an aim of at least one aspect of the present disclosure to provide an alternative inspection robot that overcomes or at least ameliorates one or more of the above-noted disadvantages.

### SUMMARY

The Applicants have found that using mechanical moving parts (e.g. paddles) for actively powered locomotion of an inspection robot carries an innate risk of damaging a GIT (or other lumen) of a patient. The present disclosure aims to provide an inspection robot capable of controlled (e.g. active) locomotion that has a minimal risk of damaging the GIT.

The present disclosure describes an inspection robot that may comprise an inspection apparatus operable to inspect an inspection site within GITs (or other lumen). The inspection apparatus may comprise at least one of: an image sensor, a balloon brush cytology apparatus, biopsy forceps, an ultrasound apparatus, and a pH sensor. The inspection apparatus may be operable to collect data and/or physical samples from the inspection site.

The inspection robot may be capable of intrinsic locomotion, meaning that all components required for locomotion (e.g. including a power source and a vibration actuator) may be within a body of the inspection robot or attached to the body of the inspection robot. Expressed differently, the inspection robot may be self-powered and capable of locomotion, which may be solely dependent on components that are part of the inspection robot.

The inspection robot may be controlled internally or externally. A processor within the inspection robot may be configured to control the inspection robot. For internal control, the processor may receive no instructions once the inspection robot enters the patient (or once a tether is detached from the inspection robot). For external control, the processor may receive instructions while the inspection robot is within the patient.

According to a first aspect of the disclosure, there is provided an inspection robot capable of locomotion comprising: a body (e.g. a soft segmented body), a vibration actuator, and a plurality of resilient legs. The plurality of resilient legs are configurable to protrude outwardly and rearwardly from the body, with respect to a direction of locomotion. Each leg is coupled to the vibration actuator at a proximal end of the leg. Each leg may be coupled directly to the vibration actuator or each leg may be coupled indirectly to the vibration actuator (e.g. through one or more intermediate parts of the inspection robot). The inspection robot may comprise one or more vibration actuators. In some embodiments, each leg may be coupled to a respective vibration actuator. In other embodiments, two or more legs may be coupled (directly or indirectly) to a common or shared vibration actuator.

The vibration actuator is operable to induce vibrations in a distal end of the leg, which serve, in use, to propel the body in the direction of locomotion by the distal end generating a pushing force against an external surface (e.g. an inner surface of the GIT or other lumen).

Thus, embodiments of the disclosure provide an inspection robot that is capable of locomotion by means of vibrating legs. The structure of the device means there are no mechanically moving parts (e.g. no mechanically moving rigid parts), which could damage a lumen. Also, as the force exerted by each leg may be small, risk of damage to the lumen can be minimised and patient discomfort limited. Conveniently, the inspection robot may be operated without complex additional control equipment and the robot may be provided as a compact and effective inspection device.

The inspection robot may be configured to generate a torque during locomotion, wherein the torque is in a plane perpendicular to the direction of locomotion. For example, the torque may be such that the inspection robot partially changes direction within a GIT that has a greater diameter than a diameter of the inspection robot (i.e. until the inspection robot encounters a GIT wall that prevents any further change of direction of the inspection robot). If the inspection robot is partially rotated within the GIT, the legs of the inspection robot may contact both sides of the GIT (i.e. on opposite sides of the inspection robot). Contacting both sides of the GIT (e.g. as a result of the inspection robot generating the torque) may enable the inspection robot to have better traction against the GIT compared to an inspection robot that does not generate such torque.

All tubular systems (i.e. GIT or other lumen) of the human body are round (i.e. have a circular cross section). However, different parts of the GIT have different diameters. Because the GIT has walls with a circular cross section, the inspection robot may always exert maximal torque on the GIT or other lumen.

The inspection robot may be configured to be asymmetric in the plane perpendicular to the direction of locomotion, and the torque may result from this asymmetry.

The inspection robot may be configured such that the plurality of legs are arranged into one or more circumferential rings about the body.

The body may comprise one or more (e.g. a plurality of) segments. The segments may be arranged in a line end-to-end.

Each segment (i.e. body segment) may comprise one ring of protruding legs. For example this may reduce a length of each segment to improve inspection robot flexibility while enabling propulsion from each segment. However, one or more segments may have a different number of rings of protruding legs compared to one or more other segments. For example, a segment may comprise no rings of legs to enable adequate space for operational components such as an inspection apparatus. A segment may comprise more than one ring of legs, for example, to increase propulsion from said segment.

Each ring of protruding legs may comprise at least three protruding legs, for example six protruding legs. Such an arrangement may provide enough contact points within the GIT to enable adequate traction, even when multiple legs are slipping or not in contact with the GIT. Rings comprising more legs may exert less force through each leg than rings comprising fewer legs. Each ring may have a different number of protruding legs. For example, each ring may comprise at least one leg. For example, each ring may comprise fewer or equal to twelve legs.

Some rings of protruding legs may have a different number of protruding legs compared to other rings of protruding legs. For example, fewer legs (e.g. zero legs) may be required on one or more parts of the body to enable adequate space for operational components such as the inspection apparatus and/or a greater number of legs may be required on one or more parts of the body to increase propulsion from the ring of protruding legs.

The protruding legs within each ring may be configured to be equidistant from one another. For example, to evenly distribute the pushing force within the GIT.

The inspection robot may be configured such that at least one of the legs on a first side of the inspection robot in a plane perpendicular to the direction of locomotion has a different value for one or more of: length, stiffness, vibration frequency, vibration amplitude, friction coefficient at the distal end; when compared to at least one of the legs on an opposing second side, so as to generate said torque.

Different values for: length, stiffness, vibration amplitude, or friction coefficient at the distal end may be used to enable a different pushing force to be generated from legs on the first side compared to legs on the opposing second side despite both the legs on the first side and the legs on the opposing second side both having vibrations induced in them by one vibration actuator (e.g. to reduce how many vibration actuators are required). Different pushing force (e.g. due to a different amplitude of vibration) may be generated from legs on the first side compared to legs on the opposing second side using one vibration actuator, if a damper is used on one side (e.g. the first side) of the inspection robot.

In some embodiments, different values for vibration frequency of the legs on the first side compared to the legs on the opposing second side may be obtained by using more than one vibration actuator (e.g. using a first vibration actuator for the first side and a second vibration actuator for the opposing second side).

The inspection robot may be configured such that the plurality of legs are arranged about the body into a first circumferential ring and a second circumferential ring, wherein the second circumferential ring is spaced in the direction of locomotion along the body from the first circumferential ring, and wherein at least one leg of the first circumferential ring and at least one leg of the second circumferential ring are offset from one another in a plane perpendicular to the direction of locomotion, so as to generate said torque.

The inspection robot may be configured such that each leg has a longitudinal axis and at least one leg has a different stiffness about the longitudinal axis in the plane perpendicular to the direction of locomotion; and/or at least one leg has a different friction coefficient about the longitudinal axis in the plane perpendicular to the direction of locomotion; so as to generate said torque.

The different stiffness about the longitudinal axis in the plane perpendicular to the direction of locomotion may result from the leg comprising a first material and a second material arranged on either side of the longitudinal axis. The first material and the second material may be arranged such that the first material is on a clockwise side of each leg and the second material is on an anticlockwise side of each leg. The first material may have a different (e.g. higher) stiffness than the second material.

The different stiffness about the longitudinal axis in the plane perpendicular to the direction of locomotion may result from a shape of the leg. For example, the leg may be curved or bent in the plane perpendicular to the direction of locomotion.

The inspection robot may be configured such that the body has a centre of mass that, for at least some duration during locomotion, is distributed asymmetrically in the plane perpendicular to the direction of locomotion; so as to generate said torque. The centre of mass may be distributed asymmetrically for only some duration if, for example, the centre of mass is rotating about an axis (e.g. due to the inspection robot comprising an eccentrically rotating mass vibration motor).

The asymmetric centre of mass may result from an off-axis mass within the inspection robot. The off-axis mass may comprise a component of the inspection robot, for example, the power source or inspection apparatus.

The inspection robot may be configured such that the vibration actuator comprises an eccentrically rotating mass vibration motor.

Expressed differently, the same eccentrically rotating mass vibration motor may be operable to generate said torque and induce the vibrations in the legs (e.g. to reduce a number of vibration actuators that are required).

The inspection robot may be configured such that the distal end of each leg is pivotable about a joint and the distal end is configurable to be behind or in front of the flexible joint with respect to a direction of locomotion.

Because the legs serve, in use, to propel the inspection robot in the direction of locomotion, and the direction of locomotion is opposite to a protrusion direction of the plurality of legs, configuring the distal ends to be behind or in front of the flexible joint enables the direction of locomotion to be changed. Expressed differently, by pivoting the distal end about the joint, the inspection robot may be configured to move in either a forward or a backward direction.

Advantageously, being able to switch the direction of locomotion may enable the inspection robot to retreat within and re-inspect areas of the GIT. For example, to hold a position within the GIT and/or retreat within the GIT for further inspection of suspect lesions.

The inspection robot may further comprise a collar actuator and a moveable collar configured to abut or be coupled to the plurality of legs; wherein the moveable collar is operable to configure the distal end of each leg to be either behind or in front of the flexible joint. The moveable collar may be moveable by the collar actuator and the collar actuator may comprise at least one of: a piezoelectric actuator, an electric screw motor, an electroactive polymer, a hydraulic actuator, a pneumatic actuator, an electromechanical solenoid, or a shape-memory alloy, a magnet. The collar actuator may be internal or external to the inspection robot.

In some embodiments, the moveable collar may be moveable to a position in which the plurality of legs extend substantially within the plane perpendicular to a direction of locomotion (e.g. substantially normal to the body). In which case, the inspection robot may remain substantially in one position, even when the vibration actuator is operating to induce vibrations in a distal end of the leg. In other words, with the distal ends of the legs positioned neither in front of or behind the flexible joint, the inspection robot can be held in position within the GIT.

The inspection robot may be a soft robot (e.g. having an outer surface that is not rigid). For example, the inspection robot may comprise highly compliant materials, similar to those found in living organisms. Expressed differently, the body may comprise soft, flexible and/or resiliently deformable materials (e.g. to prevent damage to the GIT). Preferably the vibration actuator is fully enclosed within the robot body (e.g. to prevent damage to the GIT).

An exterior of the inspection robot may be substantially contiguous. For example, the inspection robot may comprise an external sheath covering the body and the plurality of legs. The sheath may extend over flexible couplings between adjacent body parts (e.g. segments).

If the inspection robot does not comprise an external sheath, the legs may be integrally formed with the body (e.g. segments).

The inspection robot may be configured for gastrointestinal inspection and/or pipe inspection.

During locomotion, the distal end of each leg of the inspection robot may be configured to vibrate at a resonant frequency that is greater than or equal to 75 Hz, 100 Hz, 125 Hz, 175 Hz or 200 Hz. The resonant frequency is dependent on properties of the vibration actuator (e.g. size of the vibration actuator).

During locomotion, the distal end of each leg of the inspection robot may be configured to vibrate at a resonant frequency that is smaller than or equal to 185 Hz, 235 Hz, 250 Hz, or 285 Hz.

The vibration actuator may be operable to vibrate only the distal ends of each leg and not the body or other components of the inspection robot. This may be the case, for example, if the vibration actuator has a vibration frequency above a resonant frequency of the body. Having the vibration actuator operable to vibrate the distal end of each leg but not the body or other components of the inspection robot enables the inspection robot to be more power efficient as significantly less energy may be required to vibrate a small leg as opposed to a heavy body.

The vibration actuator may be in a form of a linear resonant actuator.

Advantageously, the linear resonant actuator may be used because linear resonant actuators are more power efficient than eccentric rotating mass vibration motors.

Each segment may comprise a vibration actuator. For example, so that the legs on multiple segments may be vibrated. In particular, for vibration actuators comprising a linear resonant actuator, each segment may comprise a linear resonant actuator.

Alternatively, some segments may comprise a vibration actuator and other segments may not comprise a vibration actuator. In particular, for vibration actuators comprising an eccentric rotating mass vibration motor, some segments may comprise a vibration actuator and other segments may not comprise a vibration actuator.

Each vibration actuator may be individually actuated. For example, so that a locomotion speed of the inspection robot may be controlled.

For each leg of the inspection robot, a ratio of a proximal end diameter to a distal end diameter may be greater than or equal to 4:1.

For each leg of the inspection robot, a ratio of a proximal end diameter to a distal end diameter may be smaller than or equal to 3:2.

Advantageously, the ratio between the proximal end diameter and the distal end diameter enables vibrations from the vibration actuator to transfer to the distal end efficiently, such that the pushing force generated by the distal end of the legs on the external surface is increased.

During locomotion, an angle between the distal end and a normal angle to the body may be greater than or equal to 45 degrees (e.g. greater than or equal to 45 degrees on either side of the normal angle to the body).

During locomotion, an angle between the distal end and the normal angle to the body may be smaller than or equal to 70 degrees (e.g. smaller than or equal to 70 degrees on either side of the normal angle to the body).

Advantageously, the angle between the distal end and the normal angle to the body enables the distal end of the legs to increase the pushing force generated against the external surface.

The inspection robot may comprise at least one image sensor.

An illumination apparatus—for example, one or more LEDs—may be provided to illuminate the inspection site within the GIT.

The image sensor and the illumination apparatus may be contained within a substantially dome-shaped end cap on at least one of the terminal segments.

The inspection robot may comprise a balloon operable for balloon brush cytology.

The balloon may constitute part of a balloon brush cytology apparatus. The balloon brush cytology apparatus may also comprise a deployable cover. The balloon may be switchable between an inflated state (e.g. to collect a sample of the inner surface of the lumen) and a deflated state (e.g. to enable the inspection robot to more move freely within the lumen). The deployable cover may be configured to cover the balloon (e.g. to shield the balloon in the deflated state) or to uncover the balloon (e.g. to enable the balloon to switch to the inflated state and collect a sample from the inspection site).

The legs of the inspection robot may comprise silicone (for example, because soft, resilient materials such as silicone may lessen damage to the GIT). In other embodiments, the legs may comprise other material such as polymer (e.g. medical grade polymer).

A stiffness of the legs may be greater than or equal to 5 A, 15 A, 25 A, or 35 A (i.e. in hardness durometer units). The stiffness of the legs may be smaller than or equal to 38 A, 42 A, 52 A, or 62 A.

Each segment may be joined to each adjacent segment by a flexible coupling.

The flexible couplings may be sealed to prevent ingress of material (e.g. fluid or contaminants within the GIT) into the inspection robot.

In some embodiments, a diameter of the inspection robot and/or body may be determined based on balancing a need for adequate space within the inspection robot for operational components but without making the inspection robot too large to swallow. However, in general, the inspection robot may have a diameter within a range suitable for a given application (e.g. dependent on a size or nature of the pipe/lumen requiring inspection). The body may have a diameter greater than or equal to 4 mm. The body may have a diameter smaller than or equal to 10 mm. In some embodiments, the body may have a diameter of approximately 7 mm.

In some embodiments, a length of the inspection robot may be determined based on balancing a need for adequate space within the inspection robot for operational components but without making the inspection robot too large to swallow. However, in general, the inspection robot may have a length within a range suitable for a given application (e.g. dependent on a size or nature of the pipe/lumen requiring inspection). The inspection robot may have a length greater than or equal to 20 mm. The inspection robot may have a length smaller than or equal to 44 mm. In some embodiments, the body may have a length of approximately 32 mm.

In an embodiment, the body may comprise four segments. These may provide adequate space within the inspection robot for the components but without making the inspection robot too long to swallow. However, the body may comprise any number of segments. The inspection robot may comprise at least one segment. The inspection robot may comprise fewer or equal to ten segments.

Each segment may be generally tubular or capsule-shaped, for example, so that the inspection robot does not have sharp edges that could damage the GIT.

The body may comprise one or more terminal segments (e.g. one at each end of the inspection robot) which may be longer than (e.g. twice as long as) intermediate segments. The length may provide space in the terminal segments for operational components comprising one or more: image sensor, tether connector, and/or inspection apparatus.

The inspection robot may comprise a tether. The tether may be configured to provide one or more of: power, steering instruction, and a communication channel to the inspection robot. In some embodiments, the inspection robot may be partially or substantially autonomous, for example, the inspection robot may comprise an on-board power supply and/or may be configured for wireless communication. The wireless communication may be radio frequency (RF) communication. The wireless communication may be electric field propagation (EGFP) communication. The wireless communication may be used to send/receive instructions to/from the processor within the inspection robot while the inspection robot is within the patient.

The tether may be detachable. The tether may be configured to be detachable when the image sensor shows that the inspection robot has negotiated a pyloric sphincter of a stomach and has entered a duodenum. The tether may be detachable via a controllable magnetic mechanism, enabling withdrawal of the tether from the patient through the patient's mouth.

The tether may be disconnected from the inspection robot in response to a predetermined signal. For example, a pH sensor may be employed to detect a transition from an acidic stomach environment to an alkaline duodenal environment. The pH sensor may be used to trigger separation of the tether at a pH transition point.

Alternatively, or in addition, an optical sensor, for example the image sensor, may be used to detect passage of the inspection robot through the pyloric sphincter and trigger separation of the tether once the inspection robot is through the pyloric sphincter.

Instead of having a tether, the inspection robot may be provided as an autonomous tether-free unit.

A power source may be, for example, a battery, which may be rechargeable. Each segment may be independently powered and comprise its own power source.

Advantageously, if the inspection robot is provided as an autonomous tether-free unit, the power source will have enough capacity to power the inspection robot for at least a duration of its passage through the oesophagus, stomach and small intestine.

The power source may have enough additional capacity to be powered for a subsequent passage through the colon.

The detachable tether may provide power for an initial passage of the inspection robot through the oesophagus and stomach; thereby reducing a required capacity of an on-board power source.

The inspection robot may use a power saving algorithm to prolong a useable duration of the power source (e.g. while the inspection robot is within the GIT).

The above summary is intended to be merely exemplary and non-limiting. The disclosure includes one or more corresponding aspects, embodiments or features in isolation or in various combinations whether or not specifically stated (including claimed) in that combination or in isolation. It should be understood that features defined above in accordance with any aspect of the present disclosure or below relating to any specific embodiment of the disclosure might be used, either alone or in combination with any other defined feature, in any other aspect or embodiment or to form a further aspect or embodiment of the disclosure.

### BRIEF DESCRIPTION OF THE PREFERRED EMBODIMENTS

These and other aspects of the present disclosure will now be described, by way of example only, with reference to the accompanying drawings, wherein:

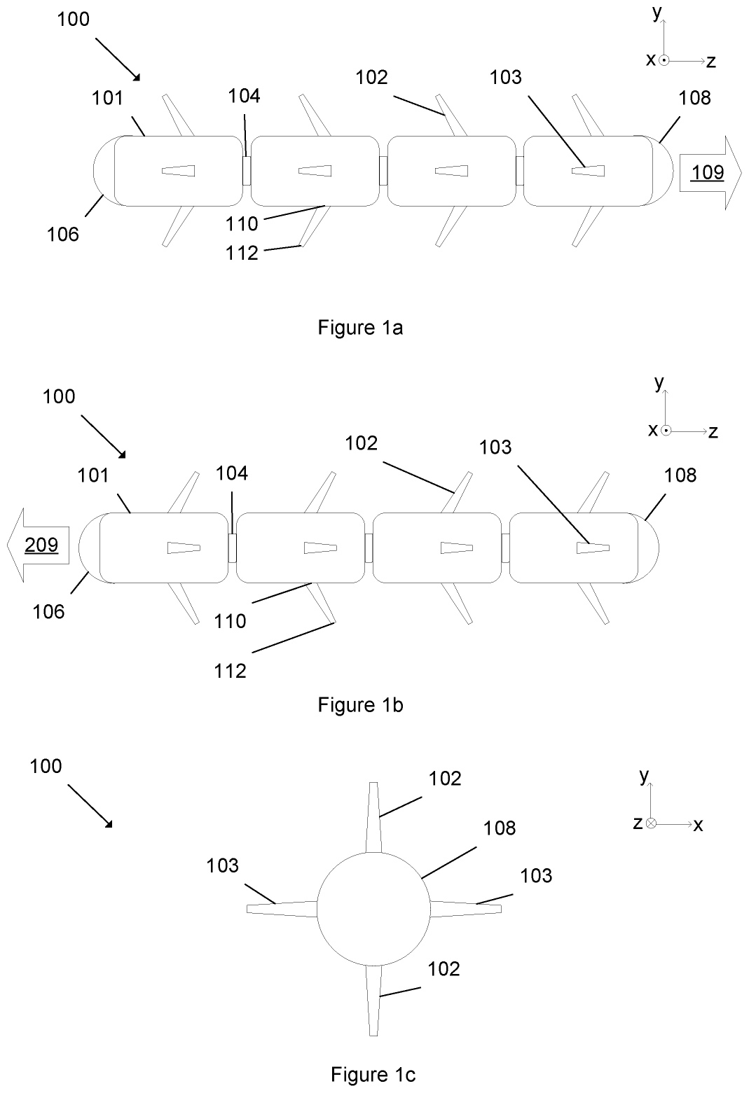

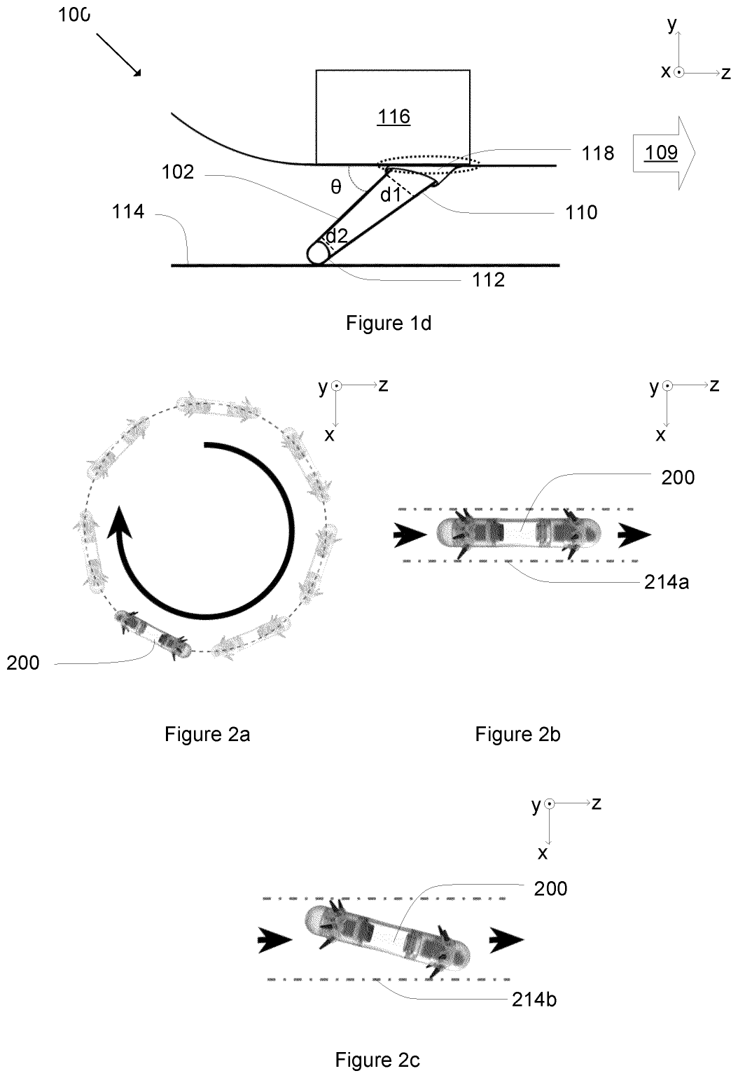

FIG. 1a depicts a side view of an inspection robot, according to a first embodiment of the disclosure, in a first configuration;

FIG. 1b depicts a side view of the inspection robot, according to the first embodiment of the disclosure, in a second configuration;

FIG. 1c depicts a front view of the inspection robot, according to the first embodiment of the disclosure;

FIG. 1d depicts a partial side sectional view through the inspection robot, according to the first embodiment of the disclosure;

FIG. 2a depicts a top view of an inspection robot configured to generate a torque during locomotion, according to a second embodiment of the disclosure that is in an unconstrained space;

FIG. 2b depicts a top view of the inspection robot configured to generate the torque during locomotion, according to the second embodiment of the disclosure that is constrained within a narrow lumen;

FIG. 2c depicts a top view of the inspection robot configured to generate the torque during locomotion, according to the second embodiment of the disclosure that is constrained within a wide lumen;

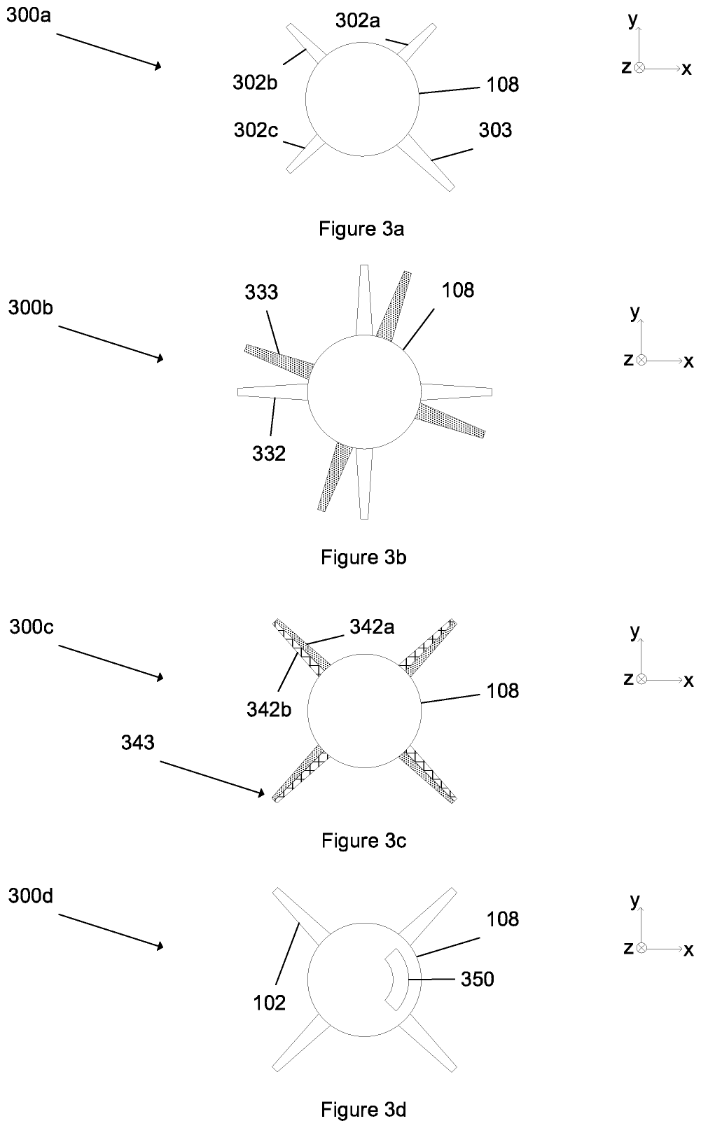

FIG. 3a depicts a front view of an inspection robot, according to a third embodiment of the disclosure;

FIG. 3b depicts a front view of an inspection robot, according to a fourth embodiment of the disclosure;

FIG. 3c depicts a front view of an inspection robot, according to a fifth embodiment of the disclosure;

FIG. 3d depicts a front view of an inspection robot, according to a sixth embodiment of the disclosure;

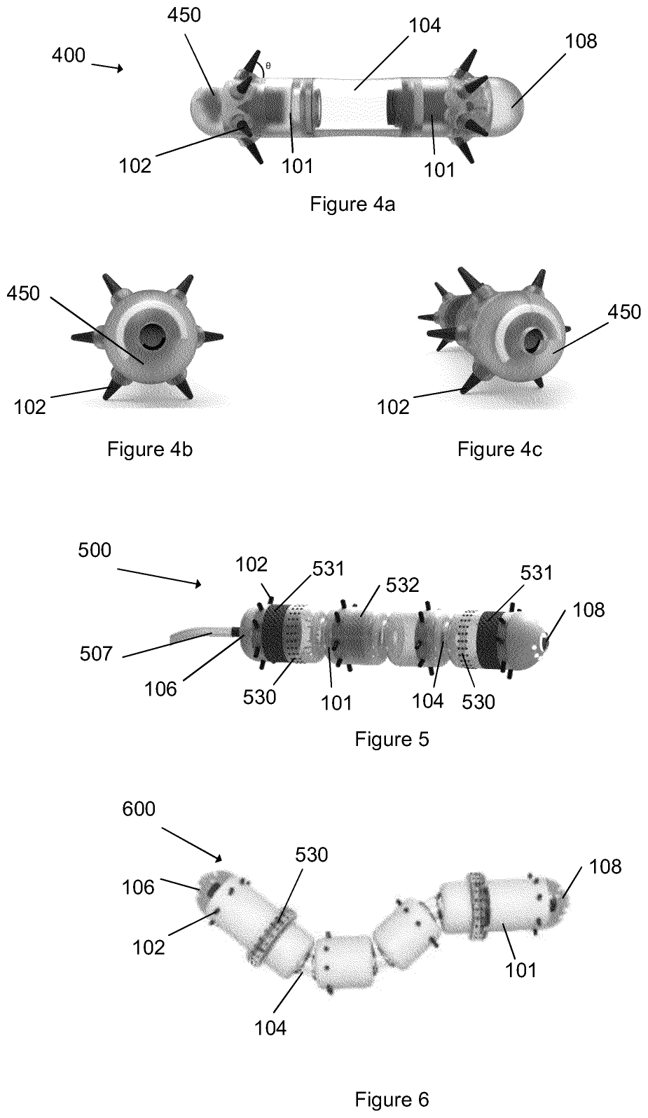

FIG. 4a depicts a side view of an inspection robot, according to a seventh embodiment of the disclosure;

FIG. 4b depicts a front view of the inspection robot, according to the seventh embodiment of the disclosure;

FIG. 4c depicts an isometric view of the inspection robot, according to the seventh embodiment of the disclosure;

FIG. 5 depicts an isometric side view of an inspection robot, according to an eighth embodiment of the disclosure;

FIG. 6 depicts a top view of an inspection robot, according to a ninth embodiment of the disclosure, in a bent configuration;

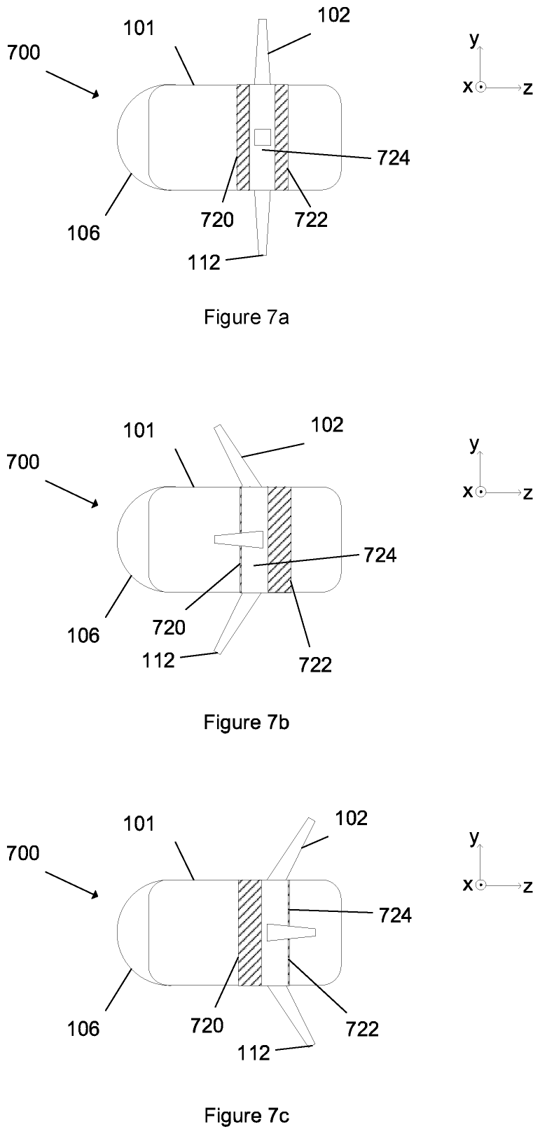

FIG. 7a depicts a partial side view of an inspection robot, according to a tenth embodiment of the disclosure, in a first configuration;

FIG. 7b depicts a partial side view of the inspection robot, according to the tenth embodiment of the disclosure, in a second configuration;

FIG. 7c depicts a partial side view of the inspection robot, according to the tenth embodiment of the disclosure, in a third configuration.

### DETAILED DESCRIPTION OF THE PREFERRED EMBODIMENTS

FIG. 1a depicts a side view of an inspection robot 100, according to a first embodiment of the disclosure, in a first configuration in which the inspection robot 100 is depicted moving in a direction of locomotion 109 that is along a positive z axis direction, as indicated.

The inspection robot 100 comprises a body having four tubular segments (i.e. body segments) 101 arranged in a line end-to-end along the z-axis. Each segment 101 is joined to each adjacent segment 101 by a flexible coupling 104. Each segment 101 comprises four resilient legs 102, 103 that are arranged to protrude outwardly and rearwardly from the body with respect to the direction of locomotion 109. The legs 102, 103 are equidistantly spaced around a circumference of the segment 101 and, as such, only three of the legs 102, 103 are visible on each segment 101, as illustrated.

Each segment 101 comprises a vibration actuator which is internal of the body and not visible in FIG. 1a. Each leg 102, 103 is coupled to the vibration actuator at a proximal end 110 of the leg and the vibration actuator is operable to induce vibrations in a distal end 112 of the leg. The vibrations induced in the distal end 112 of the leg serve, in use, to propel the inspection robot 100 in the direction of locomotion 109 by the distal end 112 generating a pushing force against an external surface (not shown).

The rearward terminal segment (i.e. the segment furthest along a negative z direction) comprises a tether connector point 106.

The forward terminal segment (i.e. the segment furthest along the positive z direction) comprises an image sensor 108.

FIG. 1b depicts a side view of the inspection robot 100, according to the first embodiment of the disclosure, in a second configuration. The second configuration is identical to the first configuration, except that the inspection robot 100 is depicted moving in an opposite direction of locomotion 209 to the direction of locomotion 109 of the first configuration (i.e. a direction of locomotion 209 that is along the negative z axis direction), and the resilient legs 102, 103 are arranged to protrude outwardly and rearwardly from the body with respect to the direction of locomotion 209.

FIG. 1c depicts a front view of the inspection robot 100, according to the first embodiment of the disclosure. FIG. 1c depicts that the four equidistantly spaced resilient legs 102, 103 protrude radially from the tubular segment 101 (not shown) that is behind the image sensor 108.

FIG. 1d depicts a partial side sectional view through the inspection robot 100, according to the first embodiment of the disclosure. FIG. 1d depicts a vibration actuator 116 and the leg 102 that is coupled to the vibration actuator at a proximal end 110 of the leg by a flexible joint 118.

The flexible joint 118 is depicted as being connected to an outer surface of the segment 101. The vibration actuator 116 is depicted as being connected to an inner surface of the segment.

The leg 102 is shown at an angle θ relative to the negative z axis direction (i.e. the positive z axis direction is depicted as being along the direction of locomotion 109). θ=90 degrees corresponds to the leg 102 protruding radially outwards from the tubular segment 101 (e.g. at a normal angle to the body). The distal end 112 of the leg 102 is pivotable about the joint 118 and the distal end 112 is configurable to be behind (e.g. 0<θ<90) or in front (e.g. 90<θ<180) of the flexible joint 118 with respect to the direction of locomotion 109. During forward locomotion, 45<θ<70. During rearward locomotion, 135<θ<160. The inspection robot does not have significant forward or rearward locomotion when 80<θ<100.

The proximal end 110 of the leg 102 has a diameter d1 and the distal end 112 of the leg 102 has a diameter d2. The diameter d1 is greater than the diameter d2. The ratio of d1 to d2 may be greater than or equal to 4:1. The ratio of d1 to d2 may be smaller than or equal to 3:2.

The vibration actuator 116 is powered by a power source (not shown) that is within the segment. The power source may be, for example, a battery. The vibration actuator 116 is controlled by a processor (not shown). The processor may be operable to switch the vibration actuator 116 between an off state and an on state. The processor may be operable to control parameters of the vibration actuator 116. For example, at least one of a vibration amplitude, a frequency, and a duty cycle.

The vibration actuator 116 may be a linear resonant actuator or an eccentric rotating mass vibration motor.

The distal end 112 of the leg 102 is shown in contact with an external surface 114. In use, the vibration actuator 116 induces vibration in the distal end 112 of the leg 102, which propels the inspection robot 100 in the direction of locomotion 109 by the distal end 112 generating a pushing force against the external surface 114.

FIG. 2a depicts a top view of an inspection robot 200 configured to generate a torque during locomotion, according to a second embodiment of the disclosure, that is in an unconstrained space.

The inspection robot 200 is similar to the first embodiment, except that: the inspection robot 200 comprises two segments 101; and each segment 101 comprises six resilient legs instead of four. The two segments 101 are terminal segments.

The inspection robot 200 is depicted on a plane containing no obstructions. The inspection robot 200 is configured to generate the torque during locomotion in a plane perpendicular to the direction of locomotion. In FIG. 2a, the torque acts in an axis of the plane, which causes the inspection robot 200 to move in a clockwise circle (i.e. the torque is directed into the plane). Expressed differently, the torque causes the direction of the locomotion of the inspection robot 200 to rotate around a rotation point.

Although the inspection robot 200 is depicted as moving in a clockwise circle, the inspection robot 200 may move in an anticlockwise circle (i.e. if the torque is directed out of the plane).

FIG. 2b depicts a top view of the inspection robot 200 configured to generate the torque during locomotion, according to the second embodiment of the disclosure, that is constrained within a narrow lumen 214a. Within the narrow lumen 214a, the inspection robot 200 experiences the torque illustrated in FIG. 2a but is unable to rotate (i.e. in the clockwise direction) due to being constrained by the narrow lumen 214a. Consequently, the direction of locomotion of the inspection robot 200 is constrained to be along a direction of the narrow lumen 214a.

FIG. 2c depicts a top view of the inspection robot 200 configured to generate the torque during locomotion, according to the second embodiment of the disclosure, that is constrained within a wide lumen 214b. FIG. 2c is similar to FIG. 2b, except that because the wide lumen 214b is wider than the narrow lumen 214a, the inspection robot 200 is able to partially rotate within the wide lumen 214b before the inspection robot 200 is constrained by the wide lumen 214b (i.e. prevented from rotating further). Because the inspection robot 200 is partially rotated within the wide lumen 214b, the legs of the inspection robot 200 contact both sides of the wide lumen 214b (i.e. on opposite sides of the inspection robot 200). Contacting both sides of the wide lumen 214b (e.g. as a result of the inspection robot 200 generating the torque) enables the inspection robot 200 to have better traction against an inner surface of the wide lumen 214b compared to an inspection robot 200 that does not generate torque.

FIG. 3a depicts a front view of an inspection robot 300a, according to a third embodiment of the disclosure. The inspection robot 300a is similar to the inspection robot 100 shown in FIG. 1a. The inspection robot 300a has four equidistantly spaced resilient legs 302a, 302b, 302c, and 303 protruding circumferentially from the tubular segment 101 (not shown) that is behind the image sensor 108.

The legs 302a, 302b, and 302c have a length that is shorter than a length of leg 303.

During use, the legs 302a, 302b, 302c, and 303 propel the inspection robot 100 in a direction of locomotion by the distal ends of the legs 302a, 302b, 302c, and 303 generating a pushing force against an external surface (not shown). Because leg 303 is longer than legs 302a, 302b, and 302c, leg 303 generates a different (e.g. larger) pushing force than legs 302a, 302b, and 302c. Consequently, during locomotion, the inspection robot 300a generates a torque which may be advantageous in increasing traction for locomotion within a lumen, as explained above.

FIG. 3b depicts a front view of an inspection robot 300b, according to a fourth embodiment of the disclosure. The inspection robot 300a is similar to the inspection robot 100 shown in FIG. 1a. The inspection robot 300a has eight resilient legs 332, 333 protruding circumferentially from the tubular segment 101 (not shown) that is behind the image sensor 108.

The legs 332, 333 are arranged about the segment 101 into a first circumferential ring of legs 332 and a second circumferential ring of legs 333. Within the first ring 332, the legs are equidistantly spaced. Within the second ring 333, the legs are equidistantly spaced. The second ring 333 is spaced in a direction of locomotion along the tubular segment 101 from the first ring 332, wherein the legs of the first ring 332 and the legs of the second ring 333 are offset from one another in a plane perpendicular to a direction of locomotion. Expressed differently, the legs of the first ring 332 are arranged to be on an anticlockwise side of each leg of the second ring 333.

During use, the legs 332, 333 propel the inspection robot 300b in the direction of locomotion by the distal ends of the legs 332, 333 generating a pushing force against an external surface (not shown). Because the legs of the first ring 332 and the legs of the second ring 333 are offset, the legs of the first ring 332 and the legs of the second ring 333 generate pushing forces in different directions. Consequently, during locomotion in the presence of a gravitational force, the inspection robot 300b generates a torque which may be advantageous in increasing traction for locomotion within a lumen, as explained above.

FIG. 3c depicts a front view of an inspection robot 300c, according to a fifth embodiment of the disclosure. The inspection robot 300c is similar to the inspection robot 100 shown in FIG. 1a. The inspection robot 300c has four equidistantly spaced resilient legs 343 protruding circumferentially from the tubular segment 101 (not shown) that is behind the image sensor 108.

Each leg 343 has a longitudinal axis within a plane perpendicular to a direction of locomotion of the inspection robot 300c. Each leg comprises a first material 342a and a second material 342b arranged on either side of the longitudinal axis. The first material 342a and the second material 342b are arranged such that the first material 342a is on a clockwise side of each leg and the second material 342b is on an anticlockwise side of each leg, as viewed. The first material 342a has a higher stiffness than the second material 342b.

During use, the legs 343 propel the inspection robot 300c in the direction of locomotion by the distal ends of the legs 343 generating a pushing force against an external surface (not shown). Because the first material 342a is stiffer than the second material 342b, for each leg 343, the first material 342a generates a different (e.g. larger) pushing force than the second material 342a. Consequently, during locomotion, the inspection robot 300c generates a torque which may be advantageous in increasing traction for locomotion within a lumen, as explained above.

FIG. 3d depicts a front view of an inspection robot 300d, according to a sixth embodiment of the disclosure. The inspection robot 300c is similar to the inspection robot 100 shown in FIG. 1a. The inspection robot 300c has four equidistantly spaced resilient legs 102 protruding circumferentially from the tubular segment 101 (not shown) that is behind the image sensor 108. FIG. 3d also depicts a mass 350.

The mass 350 is asymmetrically distributed in a plane perpendicular to a direction of locomotion, within the segment 101.

During use, the legs 102 propel the inspection robot 100 in a direction of locomotion by the distal ends of the legs 102 generating a pushing force against an external surface (not shown). Because of the asymmetric mass distribution, legs closer to the mass 350 within the plane perpendicular to the direction of locomotion generate a different (e.g. larger) pushing force than legs further from the mass 350, which generates a torque on the inspection robot 300d which may be advantageous in increasing traction for locomotion within a lumen, as explained above.

FIG. 4a, FIG. 4b, and FIG. 4c depict views of an inspection robot 400, according to a seventh embodiment of the disclosure. FIG. 4a, FIG. 4b and FIG. 4c depict a side view, a front view, and an isometric view of the inspection robot 400, respectively.

The inspection robot 400 is similar to the inspection robot 200, except that the inspection robot 400 comprises an eccentrically rotating mass vibration motor 450. The eccentrically rotating mass vibration motor 450 is arranged to be within the segment 101 that does not contain the image sensor 108. The eccentrically rotating mass vibration motor 450 is configured to rotate in a plane perpendicular to a direction of locomotion, as illustrated by the direction of the legs 102.

FIG. 5 depicts an isometric side view of an inspection robot 500, according to an eighth embodiment of the disclosure.

The inspection robot 500 is similar to the first embodiment, except that: a tether 507 is depicted attached to the tether connector point 106; a payload compartment 532 is depicted in a rearward intermediate segment 101, as illustrated; and a balloon brush cytology apparatus 530, 531 provided in both a forward terminal segment 101 and a rearward terminal segment 101.

The forward terminal segment 101 comprises the image sensor 108. Each segment comprises a plurality of resilient legs 102 coupled to a vibration actuator (not shown).

The payload compartment 532 may comprise an inspection apparatus. The payload compartment 532 may comprise a substance (e.g. drugs or other materials) delivery apparatus.

Each balloon brush cytology apparatus 530, 531 comprises a cytology balloon brush 530 and a deployable cover 531. The cytology balloon brush 530 is depicted in a deflated state. The cytology balloon brush 530 is switchable between an inflated state (e.g. to collect a sample from an inner surface of the lumen) and the deflated state (e.g. to enable the inspection robot 500 to move freely within the lumen). The deployable cover 531 is depicted as not covering the cytology balloon brush 530. The deployable cover 531 is operable to cover the cytology balloon brush 530 (e.g. to shield the cytology balloon brush 530 in the deflated state) or to uncover the cytology balloon brush 530 (e.g. to enable the cytology balloon brush 530 to switch to the inflated state and collect the sample).

FIG. 6 depicts a top view of an inspection robot 600, according to a ninth embodiment of the disclosure, in a bent configuration.

The inspection robot 600 is similar to the first embodiment, except that: a forward terminal segment 101 and a rearward terminal segment 101 are shown as being longer than two intermediate segments 101; a cytology balloon brush 530 is provided in both the forward terminal segment 101 and the rearward terminal segment 101 (i.e. as in embodiment eight); and the inspection robot 600 is in a bent configuration.

The flexible couplings 104 between each segment 101 enable the body to be bent such that each segment 101 is not aligned with an adjacent segment 101. This allows the inspection robot 600 to navigate through lumen that are bent, curved or otherwise configured, unlike if the robot had a single rigid body.

In FIG. 6, the two cytology balloon brushes 530 are depicted in an inflated state.

The forward terminal segment 101 comprises the image sensor 108. The rearward terminal segment 101 comprises the tether connection point 106. Each segment comprises a plurality of legs 102.

FIG. 7a depicts a partial side view of an inspection robot 700, according to a tenth embodiment of the disclosure, in a first configuration. The inspection robot 700 is similar to the inspection robot 100 shown in FIG. 1a.

The partial view comprises the tether connection point 106, the segment 101, a moveable collar 724, a circumferential recessed area (defined between sides 720 and 722), and four resilient legs 102 that are arranged to protrude outwardly and radially with respect to the segment 101 from a flexible joint (not shown).

A longitudinal axis of the segment 101 is along a z axis direction.

The moveable collar 724 abuts the legs 102 and is operable to configure the distal end 112 of each leg 102 to be behind or in front of the flexible joint (i.e. in the z axis direction) or radially outward from the body.

The moveable collar 724 is moveable along the longitudinal axis of the segment 101 within the recessed area. A side of the recessed area closest to a rearward end of the segment 101 (e.g. furthest along a negative z direction and closest to the tether connection point 106) is labelled 720. A side of the recessed area closest to a forward end of the segment 101 (e.g. furthest along a positive z direction and opposite the rearward end of the segment) is labelled 722.

In the first configuration, the moveable collar 724 is centred between the first side 720 of the recessed area and the second side 722 of the recessed area. In the first configuration, the legs 102 protrude radially outwardly.

During locomotion (e.g. while vibrations are being induced into the distal end 112 of the legs 102), such radially protruding legs 102 do not generate more friction in one direction (e.g. in a positive z axis direction) compared to another opposite direction (e.g. in a negative z axis direction). As such, the inspection robot 700 in the first configuration will remain relatively stationary.

FIG. 7b depicts the partial side view of the inspection robot 700, according to the tenth embodiment of the disclosure, in a second configuration.

The second configuration is similar to the first configuration, except that the moveable collar 724 has been moved to abut the rearward end 720 of the recessed area (i.e. moved away from the forward end 722 of the recessed area).

Because the moveable collar 724 abuts the legs 102, the distal ends 112 of the legs 102 are forced to move behind the flexible joint (i.e. closer to the rearward end of the segment 101).

During locomotion (e.g. while vibrations are being induced into the distal end 112 of the legs 102), such rearwardly protruding legs 102 generate more friction in one direction (e.g. in the positive z axis direction) compared to another opposite direction (e.g. in the negative z axis direction). This enables the inspection robot 700 to move in a direction of locomotion along the positive z axis direction while vibrations are being induced in the distal end 112 of the legs 102.

FIG. 7c depicts a partial side view of the inspection robot 700, according to the tenth embodiment of the disclosure, in a third configuration.

The third configuration is similar to the first configuration, except that the moveable collar 724 has been moved to abut the forward end 722 of the recessed area (i.e. moved away from the rearward end 720 of the recessed area).

Because the moveable collar 724 abuts the legs 102, the distal ends 112 of the legs 102 are forced to move in front of the flexible joint (i.e. closer to the forward end of the segment 101).

During locomotion (e.g. while vibrations are being induced into the distal end 112 of the legs 102), such forwardly protruding legs 102 generate more friction in one direction (e.g. in the negative z axis direction) compared to another opposite direction (e.g. in the positive z axis direction). This enables the inspection robot 700 to move in a direction of locomotion along the negative z axis direction while vibrations are being induced in the distal end 112 of the legs 102.

Although not shown, a collar actuator may be provided to selectively move the moveable collar in order to position the legs radially outwardly, behind or in front of the flexible joint. The collar actuator may comprise at least one of: a piezoelectric actuator, an electric screw motor, an electroactive polymer, a hydraulic actuator, a pneumatic actuator, an electromechanical solenoid, a shape-memory alloy and a magnet.

Embodiments of the disclosure provide an inspection robot that is capable of locomotion by means of vibrating legs instead of using mechanically moving parts. Advantageously, using vibrating lets reduces a risk of damaging lumen that the inspection robot is within. Another advantage of the inspection robot embodiments of this disclosure is that because each leg is small, damage to the lumen can be minimised and patient discomfort limited. Conveniently, the inspection robot may be operated without complex additional control equipment and the robot may be provided as a compact and effective inspection device.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. Furthermore, features described in relation to one embodiment may be mixed and matched with features from one or more other embodiments, within the scope of the claims.

1. An inspection robot capable of locomotion comprising:
a body, a vibration actuator, and a plurality of resilient legs;
wherein the plurality of resilient legs are arranged to protrude outwardly and rearwardly from the body, with respect to a direction of locomotion; and
wherein each leg is coupled to the vibration actuator at a proximal end of the leg and the vibration actuator is operable to induce vibrations in a distal end of the leg, which serve, in use, to propel the body in the direction of locomotion by the distal end generating a pushing force against an external surface.

2. The inspection robot of claim 1, wherein the inspection robot is configured to generate a torque during locomotion, wherein the torque is in a plane perpendicular to the direction of locomotion.

3. The inspection robot of claim 2, wherein the inspection robot is configured to be asymmetric in the plane perpendicular to the direction of locomotion, and wherein the torque results from the asymmetry; and optionally wherein at least one of the legs on a first side of the inspection robot in a plane perpendicular to the direction of locomotion has a different value for one or more of:
length,
stiffness,
vibration frequency,
vibration amplitude,
friction coefficient at the distal end;
when compared to at least one of the legs on an opposing second side, so as to generate said torque.

4. The inspection robot of claim 1, wherein the plurality of legs are arranged into one or more circumferential rings about the body; optionally wherein the plurality of legs are arranged about the body into a first circumferential ring and a second circumferential ring, wherein the second circumferential ring is spaced in the direction of locomotion along the body from the first circumferential ring, and wherein at least one leg of the first circumferential ring and at least one leg of the second circumferential ring are offset from one another in a plane perpendicular to the direction of locomotion, so as to generate said torque.

5.-6. (canceled)

7. The inspection robot of claim 3, wherein each leg has a longitudinal axis and at least one leg has a different stiffness about the longitudinal axis in the plane perpendicular to the direction of locomotion; and/or at least one leg has a different friction coefficient about the longitudinal axis in the plane perpendicular to the direction of locomotion; so as to generate said torque; or wherein the body has a center of mass that, for at least some duration during locomotion, is distributed asymmetrically in the plane perpendicular to the direction of locomotion; so as to generate said torque.

8. (canceled)

9. The inspection robot of claim 3, wherein the body comprises an eccentrically rotating mass vibration motor; so as to generate said torque; and optionally wherein the vibration actuator comprises the eccentrically rotating mass vibration motor.

10. (canceled)

11. The inspection robot of claim 1, wherein the distal end is pivotable about a joint and the distal end is configurable to be behind or in front of the flexible joint with respect to the direction of locomotion; optionally wherein the inspection robot further comprises a collar actuator and a moveable collar configured to abut or be coupled to the plurality of legs; wherein the moveable collar is operable to configure the distal end of each leg to be either behind or in front of the flexible joint; and wherein the moveable collar is moveable by the collar actuator; optionally wherein the collar actuator comprises at least one of:
a piezoelectric actuator,
an electric screw motor,
an electroactive polymer,
a hydraulic actuator,
a pneumatic actuator,
an electromechanical solenoid,
a shape-memory alloy,
a magnet.

12.-13. (canceled)

14. The inspection robot of claim 1, wherein the inspection robot is a soft robot; and/or wherein the inspection robot is configured for gastrointestinal inspection and/or pipe inspection.

15. (canceled)

16. The inspection robot of claim 1, wherein, during locomotion, the distal end is configured to vibrate at a resonant frequency that is greater than or equal to one of: 75 Hz, 100 Hz, 125 Hz, 175 Hz, or 200 Hz.

17. The inspection robot of claim 1, wherein, during locomotion, the distal end is configured to vibrate at a resonant frequency that is smaller than or equal to one of: 185 Hz, 235 Hz, 250 Hz, or 285 Hz.

18. The inspection robot of claim 1, wherein the vibration actuator comprises a linear resonant actuator.

19. The inspection robot of claim 1, wherein a ratio of a proximal end diameter to a distal end diameter for each leg is greater than or equal to 4:1; or wherein a ratio of a proximal end diameter to a distal end diameter for each leg is smaller than or equal to 3:2.

20. (canceled)

21. The inspection robot of claim 1, wherein, during locomotion, an angle between the distal end and a normal angle to the body is greater than or equal to 45 degrees; and/or wherein, during locomotion, an angle between the distal end and a normal angle to the body is smaller than or equal to 70 degrees.

22. (canceled)

23. The inspection robot of claim 1, wherein the inspection robot comprises at least one image sensor; and/or wherein the inspection robot comprises a balloon operable for balloon cytology.

24. (canceled)

25. The inspection robot of claim 1, wherein the legs comprise silicone.
